(19) United States
(12) Patent Application Publication
(10) Pub. No.: US 2014/0000632 A1
(43) Pub. Date: Jan. 2, 2014

(54) ADHESIVE STRIP WITH NON-ADHESIVE BAND AND METHOD TO REDUCE MOUTH BREATHING USING THE SAME

(71) Applicant: Somnics, Inc., Hsinchu (TW)

(72) Inventors: Chung-Chu CHEN, Hsinchu (TW); Yung-Jing HSIAO, Hsinchu (TW)

(21) Appl. No.: 13/929,130

(22) Filed: Jun. 27, 2013

Related U.S. Application Data
(60) Provisional application No. 61/665,568, filed on Jun. 28, 2012.

(51) Int. Cl. A61F 5/56

(52) U.S. Cl. CPC A61F 5/56
USPC 128/848

(57) ABSTRACT

This invention provides an adhesive strip with non-adhesive band for preventing or reducing opening of a user's mouth. The adhesive strip with non-adhesive band comprises a non-adhesive band across a central portion of an adhesive side of an adhesive strip and divides the adhesive strip into an upper adhesive part and a lower adhesive part, and the non-adhesive band has a height larger than a thickness of an upper and lower lips of the user in a closed mouth state;

### FIELD OF THE INVENTION

The present invention is related to medical devices and methods capable of reducing mouth breathing during sleep. The present invention has been developed for use in patients with sleep-disordered breathing, for example, snoring and obstructive sleep apnea.

### BACKGROUND OF THE INVENTION

Snoring and other sleep-disordered breathing conditions affect millions of patients and their spouse, partners, and/or children. Snoring typically comes from open of mouth during sleeping. Mouth breathing also causes problems for Obstructive Sleep Apnea (OSA) treatment, for example, Constant Positive Airway Pressure (CPAP), generally because the air pressure could not maintain due to air leaking via the mouth.

There have been several attempts to solve this problem by using adhesive tape to cover the mouth. U.S. Pat. Nos. 5,640,974 and 5,690,121 disclosed a chin support device in the form of a U or V-shaped piece of adhesive material having a base adhered to the user's chin below the bottom lip and two arms which extend up the face towards the cheeks to support the user's lower lip upwardly against the user's upper lip. However, the two arms are too remote from the center of the mouth and are ineffective to close the mouth without air leakage. The large area of the tape also makes it less comfortable and more noticeable.

A variation of the approach is disclosed in U.S. Pat. No. 6,089,232. The patent discloses an anti snoring device in the form of an elastic fabric panel, having adhesive covering one surface and a narrow slit cut through the panel near the center of the panel. A further variation of the approach is disclosed in U.S. Pat. No. 7,032,598 which disclosed a method comprising affixing a strip having an adhesive backing, extending from under the user's chin, over the center of the mouth. These devices could prevent the mouth from opening more effectively. However, due to the nature of their designs, the adhesive will contact over the upper and lower lips. Because the lip skin is thinner and more vulnerable, the adhesive may cause dry, cracked and peeling lips. It might even lead to bleeding if the adhesive pulls the dry skin off the lips.

### SUMMARY OF THE INVENTION

Therefore, it is one object of the present invention to provide alternative and improved methods and devices for reducing mouth breathing while substantially overcome one or more of the prior art deficiencies.

It is another object of the present invention to provide a device, which is more comfortable and will prevent peeling lips.

Yet a further object of the present invention is to provide a device, which is flexible and will allow mouth breathing, coughing or speaking during usage.

The present invention provides devices and methods for preventing or reducing the mouth opening during sleep by applying a partial-adhesive tape on the mouth to pull the upper lip and lower lip together to close and seal the mouth.

In a preferred embodiment, the present invention comprises of an adhesive strip with a non-adhesive band whereas, when applied, the non-adhesive band is aligned and centered with the center line between upper and lower lips of the user.

In another embodiment, the present invention comprises of an adhesive strip with a non-adhesive band whereas the non-adhesive band has a height about or higher than a combined thickness of the upper and lower lips in mouth-closed condition. Because the non-adhesive band will not adhere to the lips' skin, it allows interfacial movement (sliding) between the contact surfaces of the non-adhesive band and the lips' skin. Therefore, the device will not pull the lips' skin when the user try to move the lips and open the mouth, and it will not cause dry, cracked or peeling of lips.

In yet another embodiment, the present invention comprises of an adhesive strip with a non-adhesive band whereas the non-adhesive band is flexible and allows the lips to move more freely when the user intends to open the mouth to breathe, cough or speak.

In still another embodiment, the present invention comprises of an adhesive strip with a non-adhesive band whereas the non-adhesive band has a low-friction surface that allows the lips to move more freely when the user intends to open the mouth to breathe, cough or speak.

In yet another embodiment, the present invention comprises of an adhesive strip with a non-adhesive band whereas the non-adhesive band has a hydrophobic surface that repels liquid (like saliva) and reduces the chance that liquid enters the interface between the adhesive strip and reduces the adhesiveness.

In still another embodiment, the present invention comprises of a small adhesive strip with a non-adhesive band whereas the non-adhesive band only blocks part of the opening of the mouth, upper and lower lips, and is preferably configured to offset from the central region of the mouth. This may allow the user to drink more easily even when the present adhesive strip is attached to the mouth.

In yet another embodiment, the present invention provides a method for reducing mouth breathing using the present adhesive strip with non-adhesive band, comprising of: closing the user's lips tightly, aligning the non-adhesive band with a center line between closed upper and lower lips, adhering the upper adhesive part of the adhesive strip to the skin above the upper lip, and adhering the lower adhesive part of the adhesive strip to the skin below the lower lip.

### BRIEF DESCRIPTION OF THE DRAWINGS

The objects, spirits and advantages of the preferred embodiments of the present invention will be readily understood by the accompanying drawings and detailed descriptions, wherein:

FIG. 1 shows schematic diagrams of an adhesive strip with non-adhesive band according to a first embodiment of the present invention to prevent the mouth from involuntary opening;

FIG. 2 shows dimensions (relative to lips' dimension) of an adhesive strip with non-adhesive band according to the first embodiment of the present invention to prevent the mouth from involuntary opening;

FIG. 3 shows a schematic diagram of an adhesive strip with non-adhesive band according to the first embodiment of the present invention whereas the non-adhesive band has an elastic property to allow the user's mouth open partially;

FIGS. 4(A) to 4(D) show schematic cross-section diagrams of adhesive strips with various non-adhesive bands according to a second to fifth embodiments of the present invention to prevent the mouth from involuntary opening;

FIGS. 5(A) to 5(D) shows schematic cross-section diagrams of various adhesive strips with non-adhesive band according to a sixth to ninth embodiments of the present invention being applied to a user's mouth to prevent the mouth from involuntary opening;

FIGS. 6(A) to 6(E) show variations of adhesive strips with non-adhesive band according to a tenth embodiment of the present invention to prevent the mouth from involuntary opening;

FIGS. 7(A) to 7(E) show variations of adhesive strips with non-adhesive band according to an eleventh embodiment of the present invention to prevent the mouth from involuntary opening;

FIGS. 8(A) to 8(F) show variations of adhesive strips with non-adhesive band according to a twelfth embodiment of the present invention to prevent the mouth from involuntary opening;

FIGS. 9(A) to 9(D) show variations of adhesive strips with non-adhesive band according to a thirteenth embodiment of the present invention to prevent the mouth from involuntary opening;

FIGS. 10(A) to 10(D) show top, bottom, exploded, and assembly views of an adhesive strip with non-adhesive band according to a fourteenth embodiment of the present invention to prevent the mouth from involuntary opening;

FIGS. 11(A) to 11(F) show application diagrams and a method of applying an adhesive strips with non-adhesive band according to the fourteenth embodiment of the present invention to prevent the mouth from involuntary opening;

### DETAILED DESCRIPTION OF THE INVENTION

Reference will now be made in detail to the present exemplary embodiments, examples of which are illustrated in the accompanying drawings. Wherever possible, the same reference numbers are used in the drawings and the description to refer to the same or like parts.

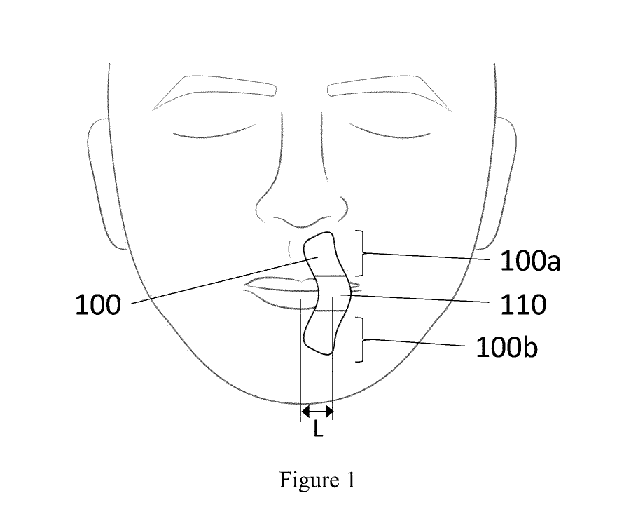

Please refer to FIG. 1. FIG. 1 demonstrates a front view of an adhesive strip 100 according to the first embodiment of the present invention. One surface of the adhesive strip 100 is adhesive. The adhesive strip 100 further comprises of a non-adhesive band 110 across a central portion of the adhesive surface of the adhesive strip 100. The non-adhesive band 110 has a height smaller than the adhesive strip 100 and an outer contour conforming to the contour of the adhesive strip 100 within their overlapping height. The non-adhesive band 110 divides the adhesive strip 100 into an upper adhesive part 100a and a lower adhesive part 100b. The upper adhesive part 100a and the lower adhesive part 100b can be temporarily attached to the skin above the user's upper lip and below the user's lower lip, respectively. The non-adhesive band 110 has a height larger than a total height of the closed lips and thus can cover the upper and lower lips that the adhesive parts, 100a and 100b, is not adhered to the lips. The vertical center line of the adhesive strip with non-adhesive band 100 is preferable having an offset L from the vertical center line of the lips. The offset L could prevent the adhesive strip 100 from blocking the central region of the mouth.

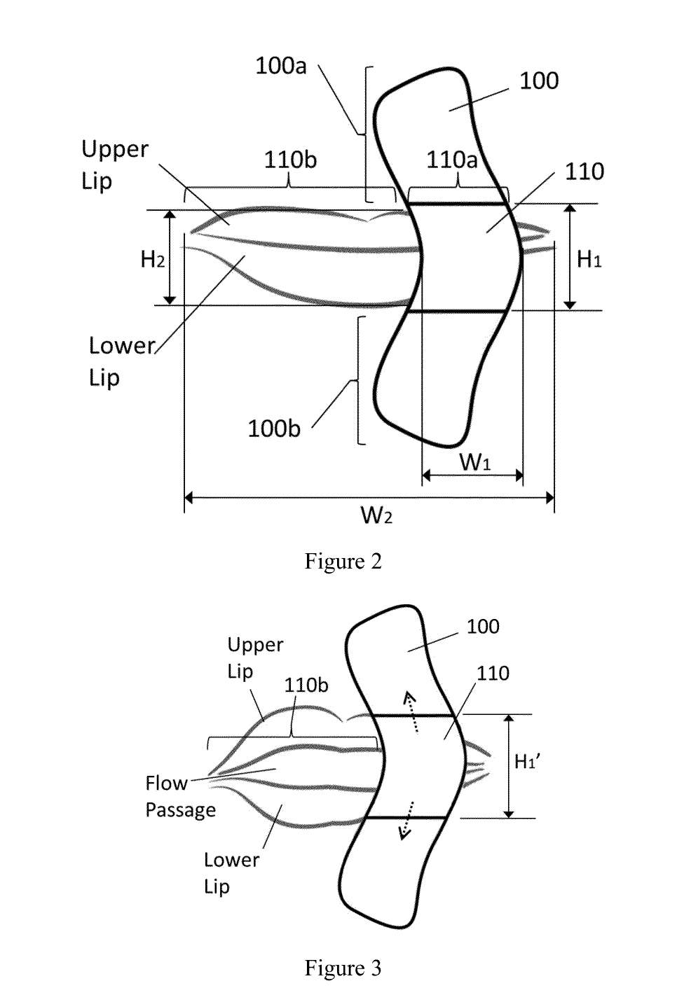

Please refer to FIG. 2. FIG. 2 demonstrates a front view of an adhesive strip with non-adhesive band according to the first embodiment of the present invention. The adhesive strip with non-adhesive band comprises of an adhesive strip 100 and a non-adhesive band 110 across a central portion of the adhesive strip 100. The non-adhesive band 110 has a width, W₁, which is smaller than the width of a user's lips, W₂, and W₁ is preferably about or smaller than ⅓ of W₂. This configuration provides an obstructive mouth opening area 110a covered by the non-adhesive band 110 besides a non-obstructive mouth opening area 110b. The non-obstructive mouth opening area 110b preserves an unblocked flow passage between the upper and lower lips when the user intends to open the mouth to breathe, cough or speak. The non-adhesive band 110 has a height H₁ which is about or higher than the combined height H₂ of the upper and lower lips in mouth-closed condition. Because the non-adhesive band will not adhere to the lips' skin, it allows interfacial movement (sliding) between the contact surfaces of the non-adhesive band 110 and the lips' skin. Therefore, the present adhesive strip with non-adhesive band will not pull the lips' skin when the user tries to move the lips and open the mouth, and it will not cause dry, cracked or peeling of lips.

Please refer to FIG. 3. FIG. 3 demonstrates a front view of an adhesive strip with non-adhesive band according to the first embodiment of the present invention while a user's mouth is opening. The adhesive strip with non-adhesive band comprises of an adhesive strip 100 and a non-adhesive band 110 across a central portion of the adhesive strip 100. The non-adhesive band 110 can be made of elastic materials with various thickness and flexibility. Potential materials of the non-adhesive band 110 include polyurethane (PU), polyethylene (PE), silicone, cloth, non-woven cloth, PET, paper, as well as other flexible thin films. When the user intends to open the mouth by exerting force on muscles around the lips or on the lateral pterygoid muscles to depress the mandible (lower jaw), the elastic non-adhesive band is extended to a height H₁′ larger than its original height H₁. The mouth is opened and creates a flow passage within the non-obstructive mouth opening area 110b allows the user to breathe, cough or speak more freely. When the user relaxes (without exerting force on muscles), the elastic non-adhesive band will return to its original height H₁ and keep the mouth in closed condition again, as shown in FIG. 2.

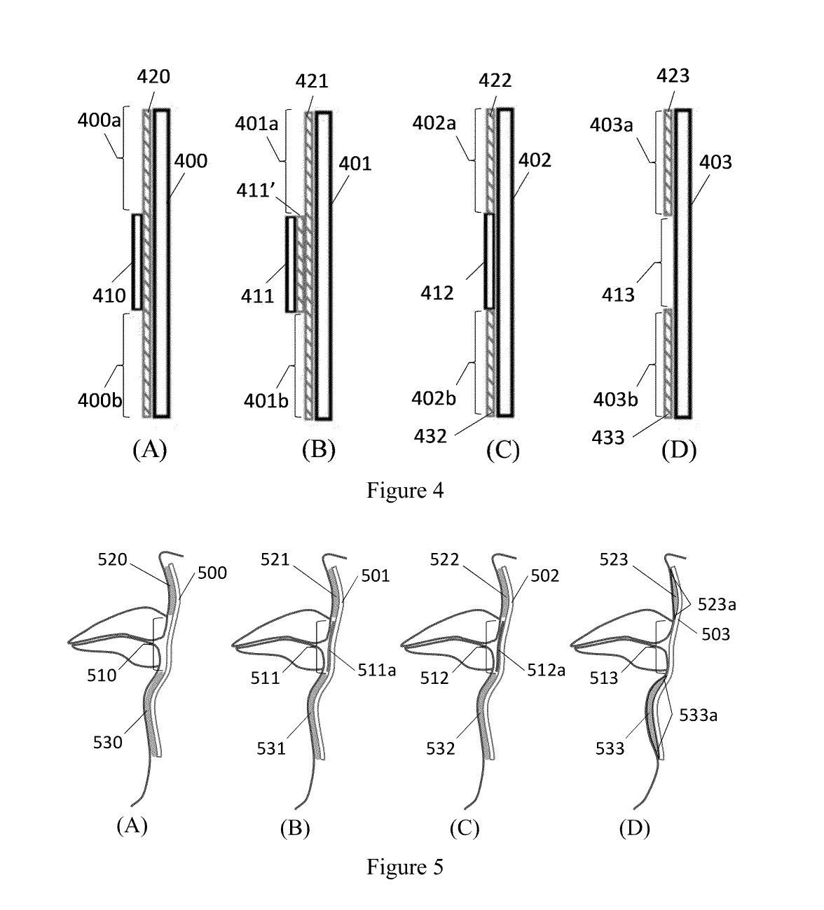

Please refer to FIGS. 4(A) to 4(D). FIG. 4(A) demonstrates a cross-section view of an adhesive strip with non-adhesive band according to a second embodiment of the present invention. In the second embodiment, an adhesive strip 400 has an adhesive layer 420 formed on its one surface and a non-adhesive band 410 which covers a central portion of the adhesive layer 420 of the adhesive strip 400. Thus the non-adhesive band 410 divides the adhesive strip 400 into an upper adhesive part 400a and a lower adhesive part 400b.

FIG. 4(B) demonstrates a cross-section view of an adhesive strip with non-adhesive band according to a third embodiment of the present invention. In the third embodiment, an adhesive strip 401 has a strip adhesive layer 421 formed on its one surface. A non-adhesive band 411 covers a central portion of the strip adhesive layer 421 of the adhesive strip 401. The non-adhesive band 411 also has a band adhesive layer 411′ on its one side facing the strip adhesive layer 421 of the adhesive strip 401. Thus the non-adhesive band 411 divides the adhesive strip 401 into an upper adhesive part 401a and a lower adhesive part 401b.

FIG. 4(C) demonstrates a cross-section view of an adhesive strip with non-adhesive band according to a fourth embodiment of the present invention. In the fourth embodiment, a partial adhesive strip 402, which has an upper strip adhesive layer 422 and a lower strip adhesive layer 432 formed on its one surface, is provided. A non-adhesive band 412 is bonded non-adhesively (such as ultrasonic bonding, thermal bonding, electrostatic bonding, etc.) to a central part of the partial adhesive strip 402 without any adhesive layer. The upper strip adhesive layer 422, the lower strip adhesive layer 432 and the non-adhesive band 412 are formed on the same surface of the partial adhesive strip 402. Thus the non-adhesive band 412 divides the adhesive strip 402 into an upper adhesive part 402a and a lower adhesive part 402b.

FIG. 4(D) demonstrates a cross-section view of an adhesive strip with non-adhesive band according to a fifth embodiment of the present invention. In the fifth embodiment, a partial adhesive strip 403, which has an upper strip adhesive layer 423 and a lower strip adhesive layer 433, is provided. A central part of the partial adhesive strip 403 without any adhesive layer forms a non-adhesive band 413. Thus the non-adhesive band 413 divides the adhesive strip 403 into an upper adhesive part 403a and a lower adhesive part 403b.

Please refer to FIGS. 5(A) to 5(D). FIG. 5(A) demonstrates a cross-section view of an adhesive strip with non-adhesive band according to a sixth embodiment of the present invention. In the sixth embodiment, a partial adhesive strip 500, which has an upper strip adhesive layer 520 and a lower strip adhesive layer 530, is provided. The lower strip adhesive layer 530 may be longer than the upper strip adhesive layer 520 to accommodate various dimensions of different user's chins. A central part of the partial adhesive strip 500 without any adhesive layer forms a non-adhesive band 510. The non-adhesive band 510 covers the upper and lower lips and the lips are untouched by the upper and lower strip adhesive layers 520 and 530 so that the upper and lower strip adhesive layers 520 and 530 will not cause drying, cracking or peeling skin of the lips.

FIG. 5(B) demonstrates a cross-section view of an adhesive strip with non-adhesive band according to a seventh embodiment of the present invention. In the seventh embodiment, a partial adhesive strip 501 having an upper strip adhesive layer 521 and a lower strip adhesive layer 531 is provided. A central part of the partial adhesive strip 501 without any adhesive layer forms a non-adhesive band 511. The non-adhesive band 511 may further comprise a low-friction surface 511a with a friction coefficient less than 0.5 that allows the lips to move more freely when the user intends to open the mouth to breathe, cough or speak.

FIG. 5(C) demonstrates a cross-section view of an adhesive strip with non-adhesive band according to an eighth embodiment of the present invention. In the eighth embodiment, a partial adhesive strip 502 having an upper strip adhesive layer 522 and a lower strip adhesive layer 532 is provided. A central part of the partial adhesive strip 502 without any adhesive layer forms a non-adhesive band 512. Moisture may cause reduction of adhesiveness of the adhesive layers and, in turn, cause the adhesive strip losing its capability to close the mouth without air leakage. The non-adhesive band 512 may further comprise a hydrophobic surface 512a that repels liquid (like saliva) and prevents it from entering the interface between the adhesive strip and the skin.

In an optional implementation, the non-adhesive band 512 may further comprise a low-friction and hydrophobic surface 512a with a friction coefficient less than 0.5 to allows the lips to move more freely when the user intends to open the mouth to breathe, cough or speak, and preventing the reduction of adhesiveness of the upper strip adhesive layer 522 and the lower strip adhesive layer 532.

FIG. 5(D) demonstrates a cross-section view of an adhesive strip with non-adhesive band according to a ninth embodiment of the present invention. In the ninth embodiment, a partial adhesive strip 503 having an upper strip adhesive layer 523 and a lower strip adhesive layer 533 is provided. A central part of the partial adhesive strip 503 without any adhesive layer forms a non-adhesive band 513. Moisture may cause reduction of adhesiveness of the adhesive layers and, in turn, cause the adhesive strip losing its capability to close the mouth without air leakage. The upper and lower strip adhesive layers (523 and 533) may further comprise thinner edges (523a and 533a) than their other portions so that the smaller thickness will reduce the contacting areas of the upper and lower strip adhesive layers (523 and 533) to absorb moisture, and liquid (like saliva) may have less chance to enter the interface between the upper and lower strip adhesive layers (523 and 533) and the skin.

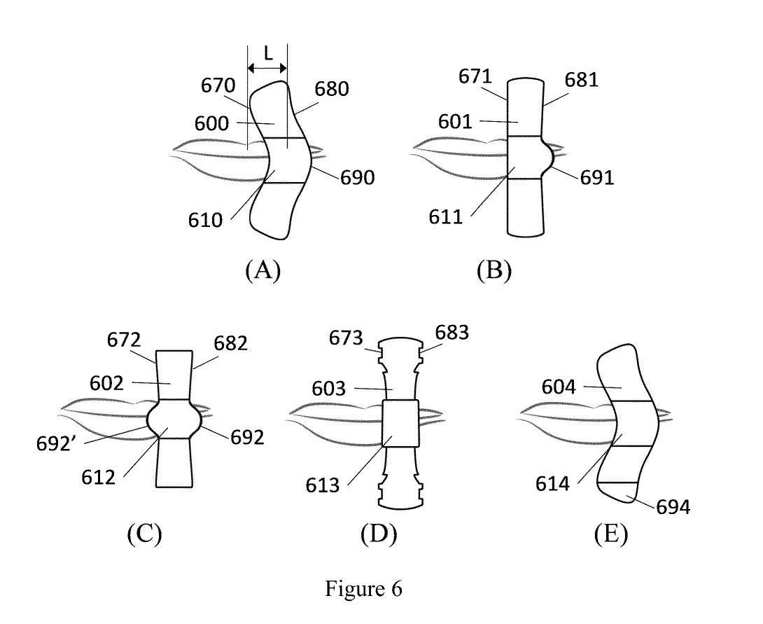

Please refer to FIGS. 6(A) to 6(D). FIGS. 6(A) to 6(D) demonstrate various adhesive strips with non-adhesive band according to a tenth embodiment of the present invention. FIG. 6(A) shows an adhesive strip with non-adhesive band which comprises of an adhesive strip 600 and a non-adhesive band 610 across a central portion of the adhesive strip 600. The adhesive strip 600 may comprise a concave edge 670 on one side and a convex edge 680 on the other side. When being applied to the mouth, the non-adhesive band 610 contacts the user's lips, and the concave edge 670 is preferably facing a central portion of the mouth while the convex edge 680 facing one of corners of the mouth. A vertical center line of the adhesive strip with non-adhesive band 600 may further an offset L from a vertical center line of the lips. The offset L is preferably about ⅓ of the lips' width. The concave-convex and offset design may fit better with the mouth's contour and leave opening at the center of the mouth. The convex edge 680 on the non-adhesive band 610 may further form a protruding tab 690, which can be used as a handler to lift the adhesive strip 600 after usage.

FIG. 6(B) shows an adhesive strip 601 with a non-adhesive band 611. The adhesive strip 601 may have unsymmetrical left edge 671 and right edge 681. The non-adhesive band 611 may have a protruding tab 691 on one of the unsymmetrical edges 671 and 681, which can be used as a handler to lift the adhesive strip 601 after usage.

FIG. 6(C) shows an adhesive strip 602 with a non-adhesive band 612. The adhesive strip 602 may have symmetrical left edge 672 and right edge 682. The non-adhesive band 612 may have two protruding tabs, 692 and 692′, on both of the symmetrical edges 672 and 682, either of which can be used as a handler to lift the adhesive strip 602 after usage.

FIG. 6(D) shows an adhesive strip 603 with a non-adhesive band 613. The adhesive strip 603 may have zigzag edges 673 and 683. The zigzag edges 673 and 683 increase contacting interface between the skin and the adhesive strip 603. The zigzag design may spread out the force applied on the adhesive strip 603 when a user tries to move the lips, and thus it may reduce the stress applied over the skin nearby and can be more comfortable.

FIG. 6(E) shows an adhesive strip 604 with a non-adhesive band 614. The adhesive strip 604 may further have a non-adhesive tab 694 on top or/and bottom edges of the adhesive strip 604, which can be used as a handler to lift the adhesive strip 604 after usage.

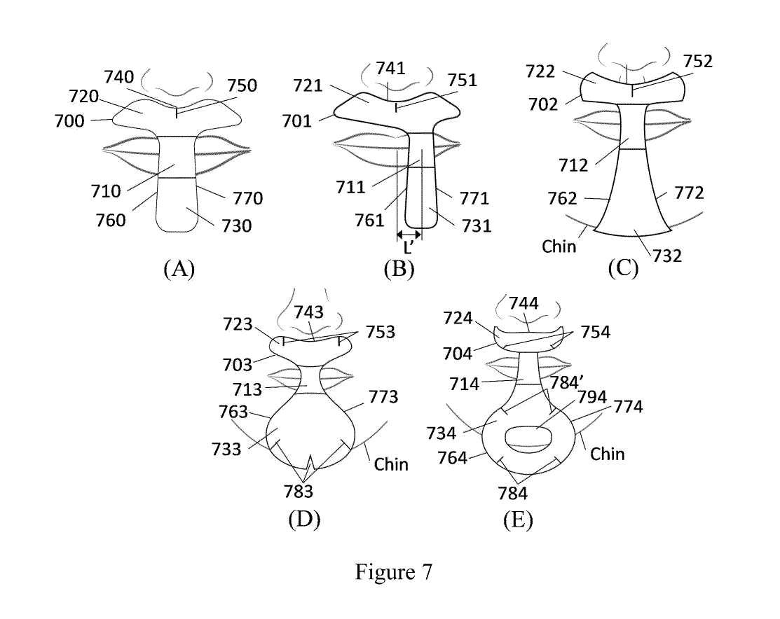

Please refer to FIGS. 7(A) to 7(E). FIGS. 7(A) to 7(E) demonstrate various adhesive strips with a parafiltrum adhesive part, a chin adhesive part and a non-adhesive band according to an eleventh embodiment of the present invention. FIG. 7(A) shows an adhesive strip 700 which comprises of a parafiltrum adhesive part 720, a chin adhesive part 730, and a non-adhesive band 710 across a mid portion of the adhesive strip 700. The parafiltrum adhesive part 720 may have a width comparable to the width of the lips to increase adhesion. The parafiltrum adhesive part 720 may further have a concave edge 740 to accommodate the shape of the nose. The parafiltrum adhesive part 720 may also have one or more cuts 750 which may improve its conformity to various contours of different user's parafiltrum. The non-adhesive band 710 is smaller than the width of the lips, and preferably about or smaller than ⅓ of the width of the lips. The chin adhesive part 730 may extend from the non-adhesive band 710 downwardly below the lips. The chin adhesive part 730 may have a width smaller than the width of the lips. The adhesive strip 700 may further have symmetrical edges 760 and 770.

FIG. 7(B) shows an unsymmetrical adhesive strip 701 which comprises of a parafiltrum adhesive part 721, a chin adhesive part 731, and a non-adhesive band 711 across a mid portion of the adhesive strip 701. The parafiltrum adhesive part 721 may further have a concave edge 741 to accommodate the shape of the nose. The parafiltrum adhesive part 721 may also have one or more cuts 751 which may improve its conformity to various contours of different user's parafiltrum. The non-adhesive band 711 is smaller than the width of the lips, and preferably about or smaller than ⅓ of the width of the lips. The chin adhesive part 731 may extend from the non-adhesive band 711 downwardly below the lips. The chin adhesive part 731 may have a width smaller than the width of the lips. The chin adhesive part 731 may have an offset L′ from the vertical center line of the lips. The offset L′ is preferably about ⅓ of the width of the lips to leave opening at the center of the mouth. The adhesive strip 700 may have unsymmetrical edges 761 and 771.

FIG. 7(C) shows another adhesive strip 702 which comprises of a parafiltrum adhesive part 722, a chin adhesive part 732, and a non-adhesive band 712 across a mid portion of the adhesive strip 702. The parafiltrum adhesive part 722 may further have a concave edge 742 to accommodate the shape of the nose. The parafiltrum adhesive part 722 may also have one or more cuts 752 which may improve its conformity to various contours of different user's parafiltrum. The non-adhesive band 712 is smaller than the width of the lips, and preferably about or smaller than ⅓ of the width of the lips. The chin adhesive part 732 may extend from the non-adhesive band 712 downwardly and reach bottom of the chin. The chin adhesive part 732 may have expanding width with diverging edges 762 and 772.

FIG. 7(D) shows another adhesive strip 703 which comprises of a parafiltrum adhesive part 723, a chin adhesive part 733, and a non-adhesive band 713 across a mid portion of the adhesive strip 703. The parafiltrum adhesive part 723 may further have a concave edge 743 to accommodate the shape of the nose. The parafiltrum adhesive part 723 may also have one or more cuts 753 on its upper and/or bottom portion which may improve its conformity to various contours of different user's parafiltrum. The non-adhesive band 713 is smaller than the width of the lips, and preferably about or smaller than ⅓ of the width of the lips. The chin adhesive part 733 may extend from the non-adhesive band 713 downwardly and reach bottom of the chin. The chin adhesive part 733 may have expanding width with convex edges 763 and 773. The chin adhesive part 733 may further have one or more cuts 783 which may improve its conformity to various contours of different user's chin.

FIG. 7(E) shows yet another adhesive strip 704 which comprises of a parafiltrum adhesive part 724, a chin adhesive part 734, and a non-adhesive band 714 across a mid portion of the adhesive strip 704. The parafiltrum adhesive part 724 may further have a concave edge 744 to accommodate the shape of the nose. The parafiltrum adhesive part 724 may also have one or more cuts 754 on its upper and/or bottom portion which may improve its conformity to various contours of different user's parafiltrum. The non-adhesive band 714 is smaller than the width of the lips, and preferably about or smaller than ⅓ of the width of the lips. The chin adhesive part 734 may extend from the non-adhesive band 714 downwardly and reach bottom of the chin. The chin adhesive part 734 may have expanding width with convex edges 764 and 774. The chin adhesive part 734 may further have multiple cuts 784 or opening(s) 794 which may improve its conformity to various contours of different user's chin.

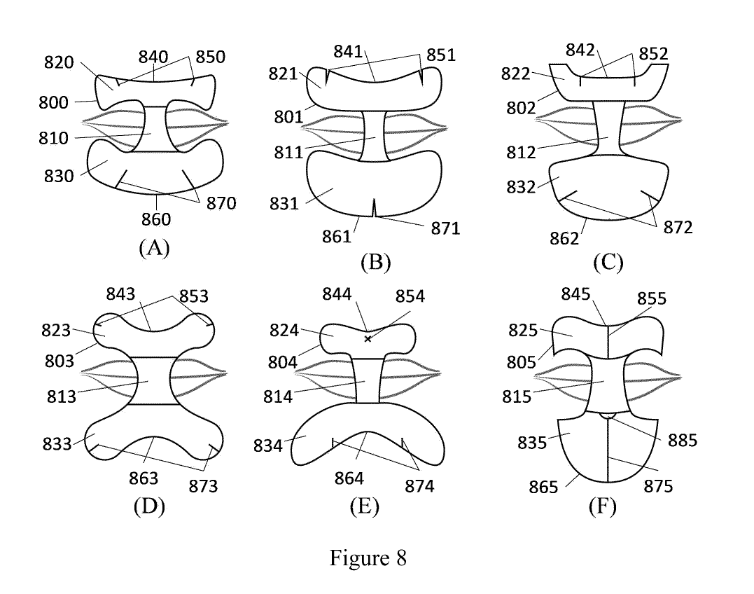

Please refer to FIGS. 8(A) to 8(F). FIGS. 8(A) to 8(F) demonstrate various adhesive strips with a parafiltrum adhesive part, a chin adhesive part and a non-adhesive part according to a twelfth embodiment of the present invention. FIGS. 8(A) to 8(F) show adhesive strips (800, 801, 802, 803, 804, and 805) comprise of a parafiltrum adhesive part (820, 821, 822, 823, 824, or 825), a chin adhesive part (830, 831, 832, 833, 834, or 835), and a non-adhesive band (810, 811, 812, 813, 814, or 815) across a central portion of the adhesive strip (800, 801, 802, 803, 804, or 805). The parafiltrum adhesive parts (820, 821, 822, 823, 824, or 825) may have a width comparable to the width of the lips to increase adhesion. The parafiltrum adhesive part (820, 821, 822, 823, 824, or 825) may further have a concave edge (840, 841, 842, 843, 844, or 845) to accommodate the shape of the nose. The parafiltrum adhesive part (820, 821, 822, 823, 824, or 825) may also have one or more cuts (850, 851, 852, 853, 854, or 855) which may improve its conformity to various contours of the parafiltrum. The non-adhesive band (810, 811, 812, 813, 814, or 815) is smaller than the width of the lips, and preferably about or smaller than ⅓ of the width of the lips. The chin adhesive part (830, 831, 832, 833, 834, or 835) may have a width comparable to the width of the lips. The chin adhesive part (830, 831, 832, 833, 834, or 835) may extend from the non-adhesive band (810, 811, 812, 813, 814, or 815) downwardly with convex bottom edges (860, 861, 862, and 865) or concave bottom edges (863 and 864). The non-adhesive band (810, 811, 812, 813, 814, or 815) may further have an offset (not shown) from the vertical center line of the lips to leave opening at the center of the mouth. The chin adhesive part (830, 831, 832, 833, 834, or 835) may further have multiple cuts (870, 871, 872, 873, 874, or 875) or opening(s) (885) which may improve its conformity to various contours of different user's chin.

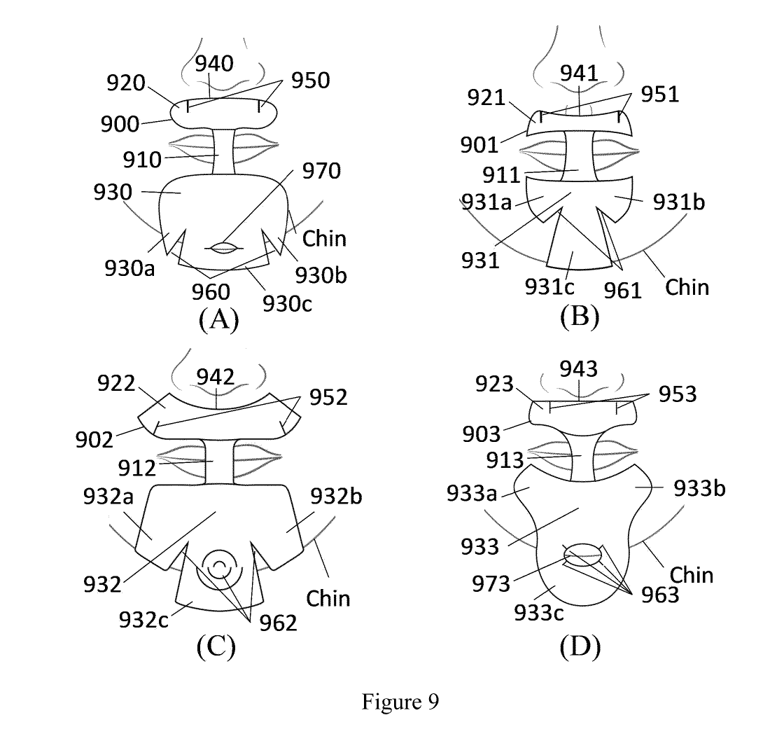

Please refer to FIGS. 9(A) to 9(D). FIGS. 9(A) to 9(D) demonstrate various adhesive strips with a parafiltrum adhesive part, a chin adhesive part and a non-adhesive part according to a thirteenth embodiment of the present invention. FIGS. 9(A) to 9(D) show adhesive strips (900, 901, 902, and 903) comprise of a parafiltrum adhesive part (920, 921, 922, or 923), a chin adhesive part (930, 931, 932, or 933), and a non-adhesive band (910, 911, 912, or 913) across a central portion of the adhesive strip (900, 901, 902, or 903). The parafiltrum adhesive parts (920, 921, 922, or 923) may have a width comparable to the width of the lips to increase adhesion. The parafiltrum adhesive part (920, 921, 922, or 923) may further have a convex edge (940), concave edge (941 or 942), or a flat edge (943) to accommodate the shape of the nose. The parafiltrum adhesive part (920, 921, 922, or 923) may also have one or more cuts (950, 951, 952, or 953) which may improve its conformity to various contours of the parafiltrum. The non-adhesive band (910, 911, 912, or 913) is smaller than the width of the lips, and preferably about or smaller than ⅓ of the width of the lips. The chin adhesive part (930, 931, 932, or 933) may have a width comparable to the width of the lips with a left extension (930a, 931a, 932a, or 933a) and a right extension (930b, 931b, 932b, or 933b). The chin adhesive part (930, 931, 932, or 933) may extend from the non-adhesive band (910, 911, 912, or 913) downwardly and reach bottom of the chin with a bottom extension (930c, 931c, 932c, or 933c). The non-adhesive band (910, 911, 912, or 913) may further have an offset (not shown) from the vertical center line of the lips to leave opening at the center of the mouth. The chin adhesive part (930, 931, 932, or 933) may further have multiple cuts (960, 961, 962, or 963) or opening(s) (973) which may improve its conformity to various contours of different user's chin.

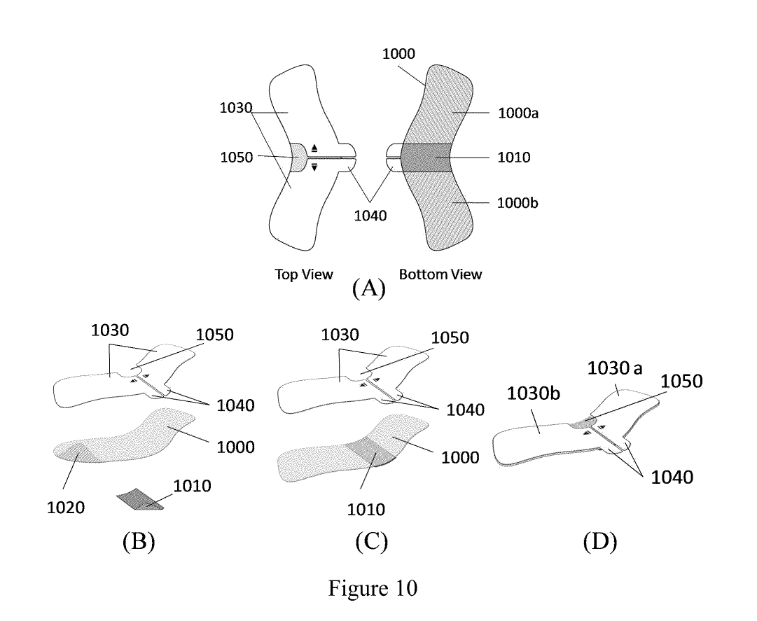

Please refer to FIGS. 10(A) to 10(D). FIGS. 10(A) to 10(D) show top, bottom, exploded, and assembly views of an adhesive strip with a non-adhesive band and a releasing support according to a fourteenth embodiment of the present invention. The fourth embodiment comprises of an adhesive strip 1000, a non-adhesive band 1010 across a central portion of the adhesive strip 1000, and one or a pair of releasing support(s) 1030 formed on a surface of the adhesive strip 1000 opposing to the non-adhesive band 1010. The non-adhesive band 1010 divides the adhesive strip 1000 into an upper adhesive part 1000a and a lower adhesive part 1000b. The releasing support 1030 may provide sheet strength to maintain the shape of the adhesive strip 1000. The releasing support 1030 may further have an applying tab 1040 near the non-adhesive band 1010 for user to hold when applying the adhesive strip 1000. The releasing support 1030 may have an aligning notch 1050 opposing to the applying tab 1040. The applying tab 1040 and the aligning notch 1050 may both be used as marks to align the non-adhesive band 1010 with the horizontal center line of the lips. The releasing support 1030 may further be divided into an upper releasing support 1030a and a lower releasing support 1030b corresponding to the upper releasing support 1030a.

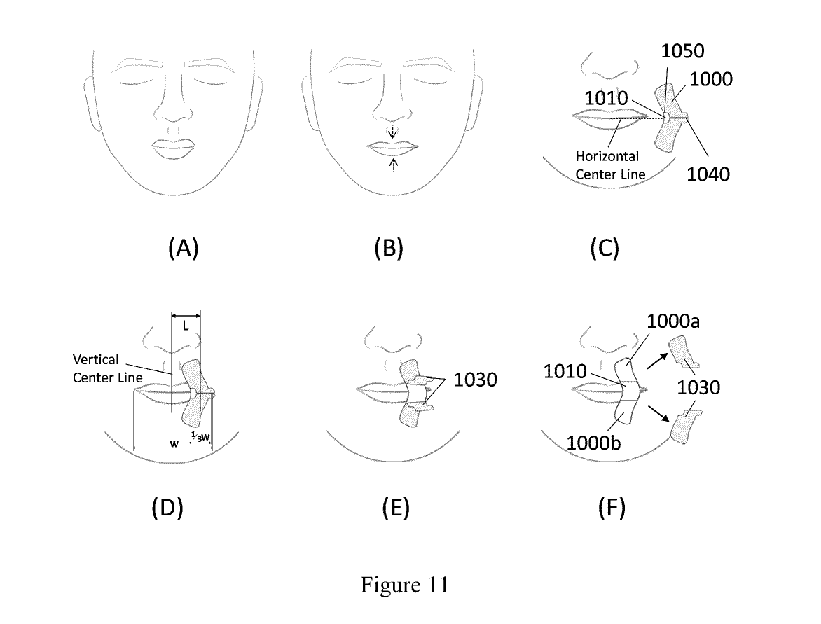

Please refer to FIGS. 11(A) to 11(F). FIGS. 11(A) to 11(D) shows application diagrams and a method of applying an adhesive strips with non-adhesive band according to the fourteenth embodiment of the present invention to prevent the mouth from involuntary opening. The method comprises closing the lips lightly by a user (FIG. 11(B)), aligning an adhesive strips with non-adhesive band 1000 with a horizontal center line of the use's lips (FIG. 11(C)) to make sure the non-adhesive band 1010 covering both upper and lower lips in a vertical direction, aligning the adhesive strips with non-adhesive band with a vertical center line of the lips with an offset L (FIG. 11(D)), attaching the upper adhesive part 1000a to the skin above the upper lip and attaching the lower adhesive part 1000b to the skin below the lower lip (FIG. 11(F)). The offset L is preferably ⅓ width of the user's lips, W. The method may further comprise aligning an aligning notch 1050 or an applying tab 1040 to the horizontal center line of the lips (FIG. 11(C)). The method may yet further comprise cover ⅓ width of the user's lips by the non-adhesive band 1010 (FIG. 11(D)). The method may still further comprise releasing one or more releasing tabs after applying the adhesive strips with non-adhesive band (FIGS. 11(E) and 11(F)) to the user's mouth. The adhesive strip used in the method may comprise an elastic non-adhesive band, which can be extended to a height larger than its original height. The mouth can be opened and creates a flow passage within the non-obstructive mouth opening area which allows the user to breathe, cough or speak more freely. When the user relaxes (without exerting force on muscles), the elastic non-adhesive band will return to its original height, and keeping the mouth in closed condition again.

The devices and methods of the present invention may further be used in combination with other apparatus for the treatment of sleep-disordered breathing. For example, the adhesive strips with non-adhesive band described herein may be used in combination with a negative oral pressure therapy device (NOPT), such as those described in, for example, U.S. Pat. No. 7,918,222 B2, filed Mar. 28, 2007, for applying negative pressure to the oral cavity. In this way, with the device of the present invention to prevent mouth from opening during sleep, and the NOPT device increases negative pressure in the oral cavity, the pressure gradient between the airway and the oral cavity can be maintained without leakage from the mouth opening, thus urging the soft palate toward the oral cavity and the tongue toward the upper hard palate to clear upper airway. In addition the adhesive strips with non-adhesive band of the present invention may be used in combination with one-way nasal valve devices, such as those described in, for example, U.S. Pat. No. 8,061,357, filed Jun. 18, 2008, and U.S. Pat. No. 8,020,700, filed Feb. 24, 2010. By increasing airway pressure using such nasal valve devices while preventing mouth leakage in the oral cavity using the present invention, the pressure gradient from airway to the oral cavity is maintained, thus urging the soft palate toward the oral cavity and the tongue toward the upper hard palate to clear upper airway. The adhesive strips with non-adhesive band of the present invention may further be used in combination with a conventional constant positive airway pressure (CPAP) apparatus for delivering air under positive pressure to the nasal airway. With the device of the present invention keeping mouth closing and preventing air leakage in the oral cavity, and the CPAP apparatus increasing Pressure in the airway, the pressure gradient between the airway and the oral cavity is maintained, thus urging soft palate toward the oral cavity and the tongue toward the upper hard palate to clear upper airway.

In addition to the specific uses described above, other embodiments and uses of the invention will be apparent to those skilled in the art from consideration of the specification and practice of the invention disclosed herein. All documents referenced herein are specifically and entirely incorporated by reference. The specification and examples should be considered exemplary only with the true scope and spirit of the invention indicated by the following claims. As will be easily understood by those of ordinary skill in the art, variations and modifications of each of the disclosed embodiments can be easily made within the scope of this invention as defined by the following claims.

We claim:

1. An adhesive strip with non-adhesive band for preventing or reducing opening of a user's mouth, comprising:
a non-adhesive band across a central portion of an adhesive side of an adhesive strip and divides the adhesive strip into an upper adhesive part and a lower adhesive part;
wherein the non-adhesive band has a height about or larger than a combined thickness of an upper and lower lips of the user in a mouth-closed condition when applied to the user's mouth.

2. The adhesive strip with non-adhesive band of claim 1, wherein the adhesive strip is applied to the mouth of the user in a way that a vertical center line of the adhesive strip with non-adhesive band has an offset distance L from a vertical center line of the user's lips.

3. The adhesive strip with non-adhesive band of claim 1, wherein the non-adhesive band has an outer contour conforming to a contour of the adhesive strip.

4. The adhesive strip with non-adhesive band of claim 1, wherein the non-adhesive band has a width W smaller than a width of the user's mouth.

5. The adhesive strip with non-adhesive band of claim 4, wherein the width W is about or smaller than ⅓ width of the user's mouth.

6. The adhesive strip with non-adhesive band of claim 1, wherein the non-adhesive band is made of elastic materials.

7. The adhesive strip with non-adhesive band of claim 1, wherein further comprises a band adhesive layer on one side of the non-adhesive band facing the adhesive side of the adhesive strip.

8. The adhesive strip with non-adhesive band of claim 1, wherein the adhesive strip is a partial adhesive strip and the non-adhesive band is bonded non-adhesively to the central part of the partial adhesive strip.

9. The adhesive strip with non-adhesive band of claim 1, wherein the adhesive strip is a partial adhesive strip and the non-adhesive band is formed on the central part of the adhesive strip without any adhesive layer in-between.

10. The adhesive strip with non-adhesive band of claim 1, wherein the lower adhesive part is longer than the upper adhesive part.

11. The adhesive strip with non-adhesive band of claim 1, wherein the non-adhesive band has a low-friction surface with a friction coefficient less than 0.5, or a hydrophobic surface, or a low-friction and hydrophobic surface with a friction coefficient less than 0.5.

12. The adhesive strip with non-adhesive band of claim 1, wherein the lower and upper adhesive parts have thinner edges than their other portions.

13. The adhesive strip with non-adhesive band of claim 1, wherein the adhesive strip has a concave edge and a convex edge;

14. The adhesive strip with non-adhesive band of claim 13, wherein the concave edge is facing a central portion of the user's mouth when applied.

15. The adhesive strip with non-adhesive band of claim 2, wherein the offset distance L is about or larger than ⅓ width of the user's mouth.

16. The adhesive strip with non-adhesive band of claim 1, wherein the adhesive strip has one kind of following edges:
unsymmetrical left and right edges;
symmetrical left and right edges; or
zigzag edges.

17. The adhesive strip with non-adhesive band of claim 1, further comprising a protruding tab formed on the non-adhesive band.

18. The adhesive strip with non-adhesive band of claim 1, further comprising a non-adhesive tab on a top or/and bottom of the non-adhesive band.

19. The adhesive strip with non-adhesive band of claim 1, wherein the upper adhesive part is a parafiltrum adhesive part with a width comparable to a width of the user's mouth and the lower adhesive part is a chin adhesive part.

20. The adhesive strip with non-adhesive band of claim 19, wherein the non-adhesive band and the chin adhesive part further have an offset to a vertical center line of the lips of the user.

21. The adhesive strip with non-adhesive band of claim 19, wherein the parafiltrum adhesive part has a concave edge, or a convex edge, or a flat edge.

22. The adhesive strip with non-adhesive band of claim 19, wherein the parafiltrum adhesive part and the chin adhesive part have one or multiple cuts.

23. The adhesive strip with non-adhesive band of claim 19, wherein the chin adhesive part has at least one opening.

24. The adhesive strip with non-adhesive band of claim 19, wherein the chin adhesive part has a width comparable to the width of the user's mouth.

25. The adhesive strip with non-adhesive band of claim 19, wherein the chin adhesive part has a concave edge, or a convex edge, or a diverging edge.

26. The adhesive strip with non-adhesive band of claim 19, wherein the chin adhesive part has at least one opening.

27. The adhesive strip with non-adhesive band of claim 19, wherein the chin adhesive part has left, right, and bottom extensions.

28. The adhesive strip with non-adhesive band of claim 1, further comprising a releasing support formed on a side of the adhesive strip opposing to the non-adhesive band.

29. The adhesive strip with non-adhesive band of claim 28, wherein the releasing support has an applying tab.

30. The adhesive strip with non-adhesive band of claim 28, wherein the releasing support has an aligning notch.

31. The adhesive strip with non-adhesive band of claim 28, wherein the releasing support comprises an upper releasing support and a lower releasing support.

32. A method for preventing or reducing a user's mouth opening, the method comprising:
applying an adhesive strip with non-adhesive band to the user's mouth, wherein the adhesive strip with non-adhesive band comprises a non-adhesive band across a central portion of an adhesive side of an adhesive strip and divides the adhesive strip into an upper adhesive part and a lower adhesive part; the non-adhesive band of the adhesive strip covers the user's upper and lower lips in a vertical direction, and the adhesive strip aligns with a vertical center line of the user's lips with an offset L; and
attaching the upper adhesive part to skins above the user's upper lip and attaching the lower adhesive part to skins below the user's lower lip;

33. The method as in claim 32, wherein the offset L is ⅓ width of the user's lips.

34. The method as in claim 32, further comprising aligning an aligning notch or an applying tab formed at one side of the non-adhesive band to a horizontal center line of the user's lips.

35. The method as in claim 32, further comprising covering ⅓ width of the user's lips by the non-adhesive band.

36. The method as in claim 32, further comprising releasing one or more releasing tabs formed on a side of the adhesive strip opposing to the adhesive band after applying the adhesive strips with non-adhesive band to the user's mouth.

37. The method as in claim 32, wherein the non-adhesive band is an elastic non-adhesive band, which can be extended to allow the user's mouth open while exerting force and closed while relaxing muscles.

38. The method as in claim 32, further comprising applying negative pressure to an oral cavity of the user.

39. The method as in claim 32, further comprising applying a one-way nasal valve device to the user to increase exhale nasal airway pressure.

40. The method as in claim 32, further comprising applying a constant positive airway pressure apparatus to the user to deliver air to the nasal airway.
